(19) United States
(12) Patent Application Publication
(10) Pub. No.: US 2026/0048273 A1
(43) Pub. Date: Feb. 19, 2026

(54) WEARABLE PHOTOTHERAPY DEVICE

(71) Applicants: Shenzhen Kaiyan Medical Equipment Co., Ltd., Shenzhen (CN); Shenzhen Light Tree Medical Technology Co., Ltd., Shenzhen (CN)

(72) Inventors: Alain Dijkstra, Amstelveen (NL); LI XIANG, Shenzhen (CN); Hu Chunlei, Shenzhen (CN); Zhou Hong, Shenzhen (CN); Peng Anle, Shenzhen (CN); Kim Laurens Markwat, Den Haag (NL); Michael Kasbergen, Schoonhoven (NL); Wang Jingjue, Shenzhen (CN); Zhu Shimin, Shenzhen (CN)

(73) Assignees: Shenzhen Kaiyan Medical Equipment Co., Ltd., Shenzhen (CN); Shenzhen Light Tree Medical Technology Co., Ltd., Shenzhen (CN)

(21) Appl. No.: 19/300,702

(22) Filed: Aug. 15, 2025

(30) Foreign Application Priority Data
Aug. 15, 2024 (CN) 202421984474X
Oct. 21, 2024 (CN) 2024225451606

(51) Int. Cl. A61N 5/06; A61N 5/067; H10H 29/01; H10H 29/24; H10H 29/85

(52) U.S. Cl. CPC A61N 5/0616; A61N 5/067; H10H 29/0364; H10H 29/24; H10H 29/857; A61N 2005/0647; A61N 2005/0652; A61N 2005/0653; A61N 2005/0654; A61N 2005/0659; A61N 2005/0663

(57) ABSTRACT

Embodiments of the present invention provide a wearable phototherapy device, comprising a flexible substrate supporting a plurality of light-emitting elements configured to emit therapeutic light of one or more wavelengths. The substrate conforms to a treatment surface and may be integrated into a mask, wrap, patch, or garment insert. A conductive thin-film layer with an integrated metal-grid structure limits current to the light-emitting elements. The elements are arranged in one or more treatment zones, each independently addressable to provide wavelength and intensity control. A connector assembly enables selective attachment and detachment of the substrate to a power and control module. The device provides uniform, targeted illumination for dermatological, cosmetic, or therapeutic applications while maintaining ergonomic comfort.

### TECHNICAL FIELD

The present invention relates to the field of non-invasive phototherapy devices or apparatuses for cosmetic, dermatological, and therapeutic applications, and more particularly to a flexible phototherapy apparatus incorporating a metal-grid current-limiting structure and micro-LED technology. The phototherapy device features a transparent, multi-layer construction that enhances heat dissipation, operational safety, and uniformity of light distribution, while maintaining a compact, lightweight, and ergonomically adaptable design.

### BACKGROUND

Wearable phototherapy devices are increasingly used in cosmetic, dermatological, and therapeutic treatments for delivering targeted light energy to the skin. These devices, which may take the form of masks, facial patches, bands, wraps, or other body-conforming structures. Such devices typically employ light-emitting sources, such as LEDs, to irradiate specific body areas with selected wavelengths for promoting skin rejuvenation, treating acne, reducing inflammation, alleviating pain, or stimulating cellular activity.

Conventional wearable phototherapy systems, however, often face several limitations. Many lack sufficient flexibility or adaptability to comfortably conform to different body contours, which can reduce treatment effectiveness due to inconsistent light exposure. Others employ bulky or rigid structures that limit portability and prolonged wear. Additionally, heat accumulation from high-intensity LEDs can cause discomfort or safety concerns, while inefficient electrical control can lead to uneven brightness or shortened device lifespan. For instance, conventional phototherapy devices typically consist of a circuit board equipped with light-emitting diodes (LEDs) and discrete resistors to limit current. These resistors are soldered onto the circuit board and used to manage current flow to the LEDs. However, such designs often lead to cluttered wiring, reduced heat dissipation efficiency, increased risk of component failure, and a bulky form factor. The widely spaced standard LEDs further result in non-uniform light coverage, leaving untreated areas on the user's face.

Furthermore, the construction of existing devices may rely on opaque or semi-opaque layers that limit the uniformity of light transmission or require complex electrical wiring arrangements that increase manufacturing complexity.

There remains a need for a wearable phototherapy apparatus that combines structural transparency, ergonomic adaptability, lightweight construction, and effective thermal management, while ensuring safe and uniform light delivery.

The present invention addresses these challenges by providing a wearable phototherapy apparatus incorporating a transparent, multi-layer structure with integrated metal-grid current-limiting technology and micro-LED light sources. This combination enables precise and uniform light output, effective thermal dissipation, and improved operational safety, while allowing the device to remain lightweight, flexible, and ergonomically adaptable. The invention can be implemented in a variety of wearable configurations, including facial masks, neck wraps, arm or leg bands, patches, or other body-conforming shapes. Similarly, the transparent structure of the phototherapy device is able to be realized through vacuum forming, molding, lamination, additive manufacturing, or other suitable fabrication techniques that support the intended transparency, flexibility, and integration of electrical components.

In addition, the invention includes a uniquely designed connector system configured to provide secure mechanical coupling, reliable electrical continuity, and ease of detachment for cleaning, replacement, or modular upgrades. The connector accommodates the flexible, multi-layer structure of the device while maintaining low-profile integration, and can be adapted for wired or wireless power supply configurations. This design supports enhanced durability, user convenience, and compatibility with a wide range of wearable phototherapy applications.

### OBJECTS OF THE INVENTION

Some of the objects of the invention are as follows:

An object of the present invention is to provide a wearable phototherapy device having plurality of micro-LEDs or micro-lasers connected with a thin film conductive ink, in order to make the device compact in size.

An object of the present invention is to provide a non-invasive, wearable phototherapy device capable of delivering controlled and uniform therapeutic light for cosmetic, dermatological, and medical applications, while ensuring improved safety, comfort, and adaptability to different anatomical regions of the body.

Another object of the invention is to incorporate a thin-film metal-grid current-limiting structure in combination with micro-LED technology, thereby enabling precise current regulation, improved thermal dissipation, enhanced operational reliability, and elimination of discrete resistors.

Another object of the present invention is to simplify the circuit layout, improve manufacturing efficiency, and reduce component failure risks by integrating the current-limiting function directly into the conductive metal-grid structure, which also serves as a heat dissipation path.

Another object of the present invention is to employ micro-LEDs arranged in a high-density matrix configuration, ensuring uniform illumination and eliminating untreated regions when applied to the skin, with adaptability for different wearable form factors such as masks, patches, wraps, or pads.

A further object of the invention is to provide a transparent or light-transmitting, multi-layer construction that ensures consistent light output, efficient heat dissipation, and a compact, lightweight, and ergonomically conformable structure.

Another object of the present invention is to offer a modular lamp-bead arrangement wherein multiple columns are connected in parallel with independent current-limiting grids, enabling localized fault tolerance, operational stability, and ease of maintenance.

It is also an object of the invention to provide a specially designed modular connector system that ensures secure electrical coupling, mechanical stability, and interchangeability between the phototherapy device and associated control modules or accessories.

Yet another object of the invention is to ensure manufacturing flexibility, allowing the device to be produced using vacuum forming, injection molding, lamination, or other fabrication methods suitable for maintaining transparency, flexibility, and structural integrity.

A further object of the present invention is to provide an ultra-thin, flexible light board assembly that may optionally be produced using a vacuum lamination process, wherein two transparent layers are bonded without adhesive by removing interstitial air, thereby reducing thickness, eliminating adhesive-related weaknesses, and improving wearing comfort.

### SUMMARY OF THE INVENTION

According to a first aspect of the present invention, a phototherapy device is provided. The phototherapy device comprising: a substrate; a plurality of light-emitting elements disposed on the substrate; and a thin film conductive structure electrically connecting the plurality of light-emitting elements, wherein the plurality of light-emitting elements is configured to emit light over a treatment area for phototherapeutic treatment.

In one embodiment of the invention, the light-emitting elements comprise micro-LEDs or laser sources.

In one embodiment of the invention, the substrate is formed of a stretchable material configured to allow stretching of the device.

In one embodiment of the invention, the thin film conductive structure comprises a zig-zag wire pattern configured to maintain circuit integrity during stretching.

In one embodiment of the invention, the substrate is made of a transparent material.

In one embodiment of the invention, the phototherapy device further comprising an outer layer and an inner layer, each formed of a flexible transparent material to provide bendability and transparency.

In one embodiment of the invention, the substrate and the outer layer are sealed together by vacuum packaging.

In one embodiment of the invention, the micro-LEDs are arranged in a matrix configuration comprising a plurality of rows and columns.

In one embodiment of the invention, the device is formed as a facial mask configured to conform to a human face, with openings for the eyes, nose, and mouth.

In one embodiment of the invention, the phototherapy device further comprising a pair of external pins configured to connect to an external controller or charging unit via magnetic or wired connection.

In one embodiment of the invention, the phototherapy device further comprising a coupling structure attached to the substrate, the coupling structure being configured to connect the phototherapy device to the external controller or charging unit.

In one embodiment of the invention, the coupling structure comprises a pair of arms, a central recess, and one or more fastening elements configured to securely mount and allow adjustable positioning of the mask.

In one embodiment of the invention, the thin film conductive structure comprises a current supply line and a metal grid current limiting structure.

In one embodiment of the invention, the metal grid current limiting structure is configured to limit current supplied to the light-emitting elements.

In one embodiment of the invention, the metal grid current limiting structure comprises a plurality of first conductive lines and a plurality of second conductive lines intersecting the first conductive lines to form a grid.

In one embodiment of the invention, the phototherapy device further comprising a welding pad formed on the thin film conductive structure, the welding pad being configured to electrically connect to an electrode of at least one of the light-emitting elements.

In one embodiment of the invention, the phototherapy device further comprising an insulating layer disposed over at least a portion of the thin film conductive structure.

In one embodiment of the invention, the thin film conductive structure comprises multiple conductive layers disposed in a stacked arrangement and electrically connected by one or more vias extending through an intermediate insulating layer.

According to a second aspect of the present invention, a phototherapy device is provided. The phototherapy device comprising: a substrate; a plurality of light-emitting elements disposed on the substrate; and a thin film conductive structure electrically connecting the plurality of light-emitting elements, wherein the thin film conductive structure comprises a metal grid current limiting structure electrically connected with at least one of the light-emitting elements, and wherein the metal grid current limiting structure is configured to limit current flow and enhance thermal dissipation.

In one embodiment of the invention, the metal grid current limiting structure comprises a plurality of first conductive lines and a plurality of second conductive lines intersecting to form a grid.

In one embodiment of the invention, each of the first conductive lines is electrically connected to at least two of the second conductive lines, and each of the second conductive lines is electrically connected to at least two of the first conductive lines.

In one embodiment of the invention, the angle formed between the first conductive lines and the second conductive lines is in the range of 60 degrees to 90 degrees.

In one embodiment of the invention, the metal grid current limiting structure is formed as a patterned metal layer on the substrate.

According to a second aspect of the present invention, a method of manufacturing a phototherapy device is provided. The method comprising: providing a flexible substrate; forming a circuit pattern for light-emitting elements; depositing a conductive material onto the flexible substrate in accordance with the circuit pattern, thereby forming a thin film conductive structure on the substrate; attaching a plurality of light-emitting elements to the thin film conductive structure; and electrically connecting the light-emitting elements to the thin film conductive structure using a conductive material.

In one embodiment of the invention, the conductive material comprises metal particles.

In one embodiment of the invention, depositing the conductive material comprises a process selected from the group consisting of screen printing, sputtering, and electroplating.

In one embodiment of the invention, the flexible substrate is selected from the group consisting of thermoplastic polyurethane (TPU).

In one embodiment of the invention, the light-emitting elements are attached using a UV-curable adhesive.

In one embodiment of the invention, the method further comprising: curing the conductive material by heating the substrate at a temperature between 100° C. and 150° C.

In one embodiment of the invention, depositing the conductive material comprises screen printing the conductive material through a stencil using a scraper.

In the context of the present specification, when an element is referred to as being “fixed to,” “mounted on,” “formed on,” or “disposed on” another element, such positioning can be direct—where the element is in physical contact with the other element—or indirect—where one or more intermediate members are positioned between the two elements. Similarly, when a component is said to be “connected,” “coupled,” or “electrically connected” to another component, the connection may be direct or may be made through intermediate conductive, insulating, or structural members.

In the context of the present specification, the use of terms such as “first,” “second,” or “third” is for descriptive purposes only and does not denote any order, priority, or relative importance. Unless explicitly stated otherwise, such terms also do not limit the quantity of features described.

In the context of the present specification, the term “plurality” refers to two or more, and the term “several” refers to more than one, unless otherwise expressly stated.

In the context of the present specification, the term “flexible substrate” refers to a base layer configured to support the mounting and interconnection of micro-LED, micro-laser, or similar light-emitting elements, while allowing the overall phototherapy device to bend, flex, or conform to non-planar or anatomical surfaces. Such substrates may include thermoplastic polyurethane (TPU), polyimide films, silicone-based elastomers, Polycarbonate (PC) or other polymeric sheets offering mechanical flexibility, durability, and biocompatibility.

In the context of the present specification, the term “wearable housing” refers to any structure or assembly configured to support and position the phototherapy device on the body. The housing may be a facial mask, headband, wrap, patch, garment insert, or other ergonomically contoured body-conforming form factor. It may be rigid, semi-rigid, or flexible, and may be formed from materials such as silicone, polyurethane, polycarbonate, or textile composites.

In the context of the present specification, the term “light source” or “phototherapy source” refers to an optical emitter such as a micro-LED, micro-laser, superluminescent diode (SLD), or equivalent device capable of emitting radiation in specific therapeutic wavelengths, including visible light, red light, and near-infrared (NIR) radiation.

In the context of the present specification, the term “micro-LED” refers to a semiconductor light-emitting device with a very small emission area, typically less than 200 μm per side, allowing for high pixel density, low power consumption, and precise control over wavelength and intensity. Micro-lasers in this context refer to miniature laser diodes or VCSELs (Vertical Cavity Surface Emitting Lasers) capable of emitting coherent light at therapeutic wavelengths.

In the context of the present specification, the term “metal-grid conductive structure” refers to a patterned conductive network formed from intersecting first and second conductive lines, configured to carry drive currents to the light sources while also functioning as a current-limiting structure to prevent overcurrent conditions and improve heat distribution. Such grids may be fabricated by printing, sputtering, electroplating, or etching conductive materials such as silver, copper, gold, or transparent conductive oxides (TCOs) onto the substrate.

In the context of the present specification, the term “transparent” refers to an optical property of the substrate, outer layer, or conductive layer that allows transmission of at least 60% of the emitted therapeutic light from the light sources through the structure to the treatment area, without substantial scattering or absorption.

In the context of the present specification, the term “connector” refers to any electrical interface, such as magnetic pogo pins, snap-fit contacts, or flexible flat cables, configured to couple the phototherapy device to an external controller, power source, or data interface. The connector may be integrated into the wearable housing and designed for quick attachment/detachment without tools.

In the context of the present specification, the term “heat dissipation” refers to design and material features that transfer or spread heat generated by the light sources to maintain skin-safe temperatures and prevent performance degradation. This may include high thermal conductivity substrates, thermally conductive encapsulants, or the metal-grid conductive structure acting as a heat-spreading layer.

Unless otherwise stated, the term “light” as used in this specification encompasses electromagnetic radiation in the visible (380-780 nm) and infrared (780 nm-1000 nm) ranges, particularly red light (620-750 nm) and near-infrared (750-1400 nm) wavelengths commonly used in photobiomodulation therapy. Particular wavelengths which may be selected as the dominant emissive wavelength may include the follow, without any preference to be indicated by order: 400 nm, 405 nm, 420 nm, 430 nm, 450 nm, 465 nm, 515 nm, 530 nm, 532 nm, 590 nm, 630 nm, 633 nm, 640 nm, 650 nm, 655 nm, 660 nm, 670 nm, 680 nm, 780 nm, 785 nm, 810 nm, 830 nm, 840 nm, 850 nm, 860 nm, 870 nm, 904 nm, 915 nm, 980 nm, 1015 nm, 1060 nm, 1065 nm, 1070 nm, 1200, and 1400 nm. As used herein, the term “light therapy” refers to the use of one or more light sources of any type that emits light with a wavelength between about 400 and 1400 nm. The device may also emit blue or ultraviolet light for surface-level treatments such as acne reduction or microbial control.

In the context of this specification, terms like “light”, “radiation”, “irradiation”, “emission” and “illumination”, etc. refer to electromagnetic radiation in frequency ranges varying from the visible frequencies to Infrared (IR) frequencies and wavelengths, wherein the range is inclusive of visible light, and IR frequencies and wavelengths. Preferably, it refers to low-level electromagnetic radiation of low-level red and near-infrared (NIR) light. It is to be noted here that IR radiation can be categorized into several categories according to respective wavelength ranges, which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm), and Far IR (15-1000 μm). In this regard, light application is at relatively low energy densities, typically below about 500 mW, as compared to other forms of laser therapy that are used for ablation, cutting, and thermally coagulating tissue. In some instances, electromagnetic radiation can also be in wavelengths in the blue or ultraviolet regions, especially for the treatment of conditions that occur at the skin surface, such as psoriasis or infection.

In the context of the specification, the term “light source” or “phototherapy source” etc. refers to a source emitting coherent laser light, or light-emitting diodes (“LEDs”). The term “light therapy” refers to light generated from any of the sources, such as lasers, LED sources, or Super luminous diodes (“SLD”).

In the context of the specification, “Light Emitting Diodes (LEDs)” refer to semiconductor diodes capable of emitting electromagnetic radiation when supplied with an electric current. The LEDs are characterized by superior power efficiencies, smaller sizes, rapid switching speeds, physical robustness, and longer lifespans compared to incandescent or fluorescent lamps. The one or more LEDs may include through-hole type LEDs (generally emitting electromagnetic radiation in red, green, yellow, blue, and white colors), Surface Mount Technology (SMT) LEDs, Bi-color LEDs, Pulse Width Modulated RGB (Red-Green-Blue) LEDs, and high-power LEDs, among others.

Materials used in one or more LEDs may vary from one embodiment to another, depending upon the frequency of radiation required. Different frequencies can be obtained from LEDs made from pure or doped semiconductor materials. Commonly used semiconductor materials include nitrides of Silicon, Gallium, Aluminum, Boron, Zinc Selenide, etc., in pure form or doped with elements such as Aluminum and Indium, etc. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider color gamut. White and other colored lights may also be produced using phosphor coatings such as Yttrium Aluminum Garnet (YAG) in combination with a blue LED to generate white light and Magnesium-doped potassium fluorosilicate in combination with a blue LED to generate red light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, a discussion on the generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety, by reference.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings wherein like reference letters and numerals indicate the corresponding parts in various figures in the accompanying drawings, and in which:

FIG. 1A illustrates a front perspective view of the structure of a phototherapy device integrated with a plurality of micro-LEDs, in accordance with an embodiment of the present invention.

FIG. 1B illustrates a perspective view of the structure of a mask-shaped phototherapy device, in accordance with an embodiment of the present invention.

FIG. 1C illustrates a perspective view of the patch structure of a phototherapy device, in accordance with an embodiment of the present invention.

FIG. 2 illustrates the methodology of making a circuit board of the phototherapy device, in accordance with an embodiment of the present invention.

FIG. 3 illustrates a circuit board of a phototherapy device, in accordance with an embodiment of the present invention.

FIG. 4 illustrates a circuit board with a point ‘A’ of the phototherapy device, in accordance with an embodiment of the present invention.

FIG. 5 illustrates an enlarged view of point ‘A’ of the circuit board of the phototherapy device, in accordance with an embodiment of the present invention.

FIG. 6 illustrates an assembly of a metal grid current limiting structure, in accordance with an embodiment of the present invention.

FIG. 7 illustrates a structure of a lamp bead circuit, in accordance with an embodiment of the present invention.

FIG. 8 illustrates a perspective view of the connection of the phototherapy device with a control box and a charging connector, in accordance with an embodiment of the present invention.

FIG. 9 illustrates a perspective view of the phototherapy device and the charging connector connected together, with a point ‘B’, in accordance with an embodiment of the present invention.

FIG. 10 illustrates an enlarged view of a point ‘B’ of the charging connector, in accordance with an embodiment of the present invention.

FIG. 11 illustrates a top perspective view of a line card of the charging connector, in accordance with an embodiment of the present invention.

FIG. 12 illustrates a front perspective view of the phototherapy device without the connection of the charging connector, in accordance with an embodiment of the present invention.

FIG. 13 illustrates a front perspective view of a first card body of the charging connector, in accordance with an embodiment of the present invention.

FIG. 14 illustrates a front perspective view of a second card body of the charging connector, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

Embodiments of the present invention disclose a wearable, non-invasive phototherapy device designed for cosmetic, dermatological, and medical applications. The invention integrates a flexible substrate, a plurality of light-emitting elements, and a thin-film conductive structure, arranged to deliver controlled, uniform, and safe therapeutic light to a treatment area. The plurality of light-emitting elements is disposed on the substrate, electrically connected via the thin-film conductive structure. In certain embodiments, the light-emitting elements are arranged in a matrix configuration comprising rows and columns, enabling full coverage of the treatment area and eliminating gaps present in conventional spaced-apart LED arrays. The invention is adaptable to multiple wearable configurations, including but not limited to facial masks, neck wraps, wristbands, patches, or joint wraps, thereby enabling treatment across diverse anatomical regions.

In an embodiment of the present invention, the wearable phototherapy device integrates an advanced light-emitting structure comprising an array of micro-light-emitting diodes (micro-LEDs) or, in a certain configuration, micro-laser emitters, disposed upon or embedded within a transparent substrate. The use of micro-LEDs or micro-lasers enables a significant reduction in device thickness, while providing high luminous efficiency, precise wavelength output, and targeted energy delivery to specific treatment zones. The reduced footprint of these emitters allows for closer packing density, resulting in improved light uniformity and better coverage across curved surfaces of the body or faces.

In one embodiment, the device comprises a flexible substrate fabricated from transparent or light-transmitting materials such as thermoplastic polyurethane (TPU), polyethylene terephthalate (PET), polycarbonate (PC), or silicone-based polymers. The substrate may be stretchable to conform to the contours of the target treatment area, and in certain embodiments is capable of elastic recovery to maintain durability after repeated bending or stretching. The transparent structure of the apparatus, achieved through materials such as polycarbonate, PET, or flexible glass, provides both aesthetic and functional advantages. Transparency allows light from the emitters to radiate evenly across the treatment area, without shadowing or occlusion caused by structural elements. In some embodiments, the transparent structure is multi-layered, incorporating separate functional layers for optical transmission, electrical conduction, thermal management, and environmental sealing. These layers may be fabricated using various methods, including but not limited to roll-to-roll printing, lamination, microfabrication techniques, and vacuum deposition processes.

In an embodiment of the present invention, the thin-film conductive structure is a metal-grid conductive film. The metal-grid conductive film is configured to supply electrical power to the light-emitting elements while also performing current-limiting and thermal dissipation functions. In one embodiment, the conductive structure includes a metal-grid current-limiting network formed of a plurality of first conductive lines and a plurality of second conductive lines intersecting at an angle between 60° and 90° to form a grid pattern. Each conductive line is electrically connected to multiple intersecting lines to enhance redundancy and maintain electrical continuity in case of localized damage. The grid may be fabricated from materials such as copper, silver, aluminium, gold, or conductive alloys, applied via screen printing, sputtering, electroplating, or other deposition techniques. In certain embodiments, multiple conductive layers may be stacked and electrically connected through vias extending through an intermediate insulating layer.

This metal-grid conductive film, formed as a fine, optically transparent grid pattern, serves as a primary electrical pathway to deliver power to the micro-LED or micro-laser array. The grid's configuration is optimized to achieve high electrical conductivity while minimizing optical interference, allowing light from the emitters to pass through with negligible obstruction. The metal-grid film also inherently supports current-limiting functionality, thereby eliminating the need for discrete resistors in the circuit. This elimination of resistors reduces component count, enhances transparency, minimizes weight, and improves heat management by removing local hotspots that resistors would otherwise generate. In an embodiment, a zig-zag or serpentine conductive wire pattern is incorporated into the metal-grid conductive film to preserve circuit integrity during elongation.

Effective heat management is achieved through a combination of the thin-film metal-grid conductor's high thermal conductivity, thermally conductive transparent substrates, and optional micro-patterned heat spreaders. In some embodiments, transparent graphene layers or thin metallic oxide films may be added to further improve heat dissipation while maintaining transparency. The uniform distribution of electrical current through the metal-grid film reduces localized heating, and the elimination of resistors prevents concentrated thermal load points. As a result, the apparatus remains comfortable for prolonged skin contact, with surface temperatures maintained within safe operating limits.

In some embodiments, welding pads are formed on the thin-film conductive structure to facilitate an electrical connection between the conductive network and the electrodes of the light-emitting elements. An insulating layer may be disposed over portions of the conductive structure to prevent short circuits, improve safety, and protect the conductive material from oxidation or wear.

In another embodiment, the device further comprises an outer layer and an inner layer, both made of flexible transparent materials, which enclose the substrate and conductive structure. These layers provide mechanical protection, environmental sealing, and optical clarity. The outer and inner layers may be bonded to the substrate via vacuum packaging, lamination, thermal bonding, adhesive or other sealing processes, ensuring an ultra-thin, compact profile while preventing ingress of moisture or contaminants.

Electrical connection to the device may be achieved through a pair of external pins, magnetic connectors, or wired couplings. In an embodiment, a specialized connector assembly is provided in the wearable phototherapy device. The connector assembly is configured for quick, reliable, and ergonomic attachment to a power source or a control module, or an external controller. The coupling assembly comprises a coupling structure or a connector, which is mounted on the substrate to provide secure attachment to an external controller or charging unit. The coupling structure may include a pair of arms, a central recess, and fastening elements such as clips, screws, or magnetic latches, which allow adjustable positioning of the device and stable mechanical support during use. Strain-relief features may be incorporated to prevent damage to the conductive traces when the connector is engaged or disengaged. The connector may be integrated into the edge of the phototherapy apparatus or positioned at a dedicated docking interface. In some embodiments, the connector includes locking features, low-profile contacts, and keyed alignment to ensure correct polarity and mechanical stability. The connector may support wired or hybrid wired-wireless operation, and in certain cases, may incorporate a magnetic coupling mechanism for rapid engagement and disconnection. Electrical connections between the connector and the metal-grid film are configured to maintain transparency and flexibility without creating visible or tactile obstructions.

Manufacturing of the device may involve providing the flexible substrate, forming a desired circuit pattern for the light-emitting elements, and depositing a conductive material onto the substrate in alignment with the circuit pattern to form the thin-film conductive structure. Suitable deposition processes include screen printing, sputtering, electroplating, or inkjet printing. The conductive material may contain metal particles and, in certain embodiments, is cured by heating the substrate to a temperature between 100° C. and 150° C. The light-emitting elements may be attached using conductive adhesives, UV-curable adhesives, soldering, or other bonding techniques, followed by electrical connection through conductive pads or traces.

In operation, the wearable phototherapy device is designed to closely conform to the natural contours of the target treatment area, such as the face, neck, scalp, joints, or other body regions, ensuring consistent and uniform delivery of phototherapeutic energy. The integrated micro-LED or micro-laser arrays are individually addressable, enabling the creation of discrete treatment zones with independently controlled wavelength outputs. This configuration allows precise customization of therapeutic protocols, accommodating diverse dermatological and clinical objectives, including anti-aging, acne management, pigmentation correction, wound healing, and pain relief. The use of a lightweight, transparent, and flexible structural substrate permits seamless integration into various wearable formats such as facial masks, wraps, headbands, adhesive patches, or garment inserts, maintaining optimal user comfort and full freedom of movement while preserving treatment efficacy.

The present invention is not limited to a particular wearable geometry, light wavelength, or manufacturing process, and may incorporate additional features such as optical diffusers, microlens arrays, thermal sensors, proximity sensors, or control circuits for regulating light output. Through the integration of a transparent flexible substrate, a metal-grid current-limiting conductive structure, and densely arranged micro-LEDs, the invention achieves improved safety, uniformity of light distribution, thermal management, and adaptability compared to conventional wearable phototherapy devices.

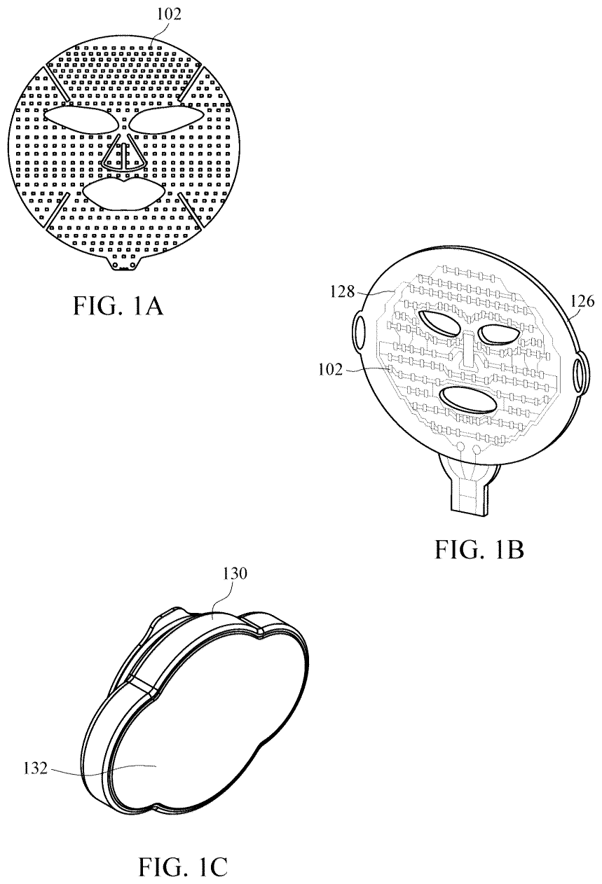

Referring to FIGS. 1A, 1B, and 1C, in accordance with an embodiment of the present invention illustrate a phototherapy device of different shapes and sizes, such as a face mask (FIG. 1B), and a patch (FIG. 1C), each incorporating a plurality of micro-LED lamp beads 102 or micro-lasers mounted on a flexible substrate with an integrated conductive film and circuit trace network.

The substrate is formed of a flexible, biocompatible material, such as thermoplastic polyurethane (TPU) or other elastomeric films, configured to conform to the contours of the intended treatment area. Disposed on the substrate is a thin conductive film, patterned to define circuit traces that electrically interconnect the micro-LEDs. These circuit traces may be formed by screen printing, sputtering, or electroplating, and are configured to maintain electrical continuity during bending or stretching of the device.

The micro-LED lamp beads 102 are directly mounted onto welding pads 120 positioned along the circuit traces. Compared to conventional LEDs, which are typically spaced at fixed intervals, the micro-LED lamp beads 102 can be positioned at significantly higher densities across the substrate surface. The micro-LEDs provide more effective therapy for the nose area compared to conventional LEDs, as their smaller size allows a higher density of LEDs to be positioned over the nose, thereby delivering more uniform and targeted treatment. This high-density arrangement ensures that virtually every smallest unit area of the treatment surface is exposed to therapeutic light, eliminating untreated gaps that can occur with standard LED spacing. This results in a substantially greater effective treatment surface area, enabling uniform light delivery and more consistent therapeutic outcomes. The phototherapy device may be configured in various wearable forms, such as a facial mask, neck wrap, wristband, patch, or joint wrap etc., allowing it to deliver treatment to different regions of the body.

Each device further includes a connector, such as a magnetic coupling or low-profile plug, electrically linked to the circuit traces for connection to an external control box or power supply. The connector provides power and control signals to the micro-LED lamp beads 102, allowing adjustment of treatment parameters such as light intensity and duration.

By integrating densely arranged micro-LED lamp beads 102 with a flexible substrate, conductive film, and precisely patterned circuit traces, the devices shown in FIGS. 1A to 1C achieve both structural adaptability to different body regions and maximized therapeutic coverage through superior utilization of the available surface area.

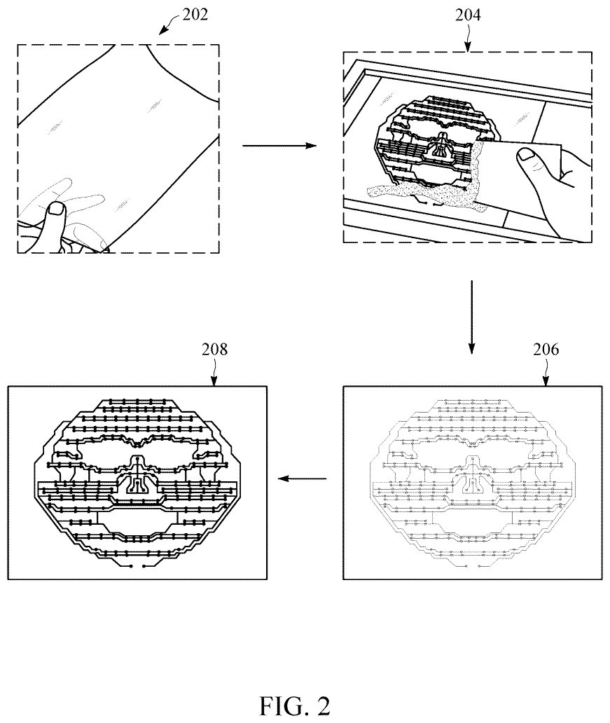

Referring to FIG. 2, an advanced method flow of manufacturing a phototherapy device is illustrated. The method enables the creation of an ultra-thin, feather-light, and highly flexible light-emitting circuit that can be worn directly on the skin with unparalleled comfort. By combining precision screen printing with novel substrate materials, this process achieves a substantial reduction in production cost while delivering superior adaptability for wearable phototherapy applications.

In the first step 202, the carefully engineered circuit layout for the micro-LED array is digitally converted into a high-resolution screen printing stencil. The stencil is not only a simple template, but a precision-engineered tool that defines the exact position, geometry, and continuity of every conductive trace and welding pad 120 in the circuit. The screen ensures micron-level accuracy, enabling an intricate electrical network capable of accommodating a dense grid of micro-LED lamp beads 102 while preserving uniform current distribution across the entire treatment surface.

The process then involves substrate preparation, which is a critical factor in determining the final flexibility and performance of the device. In an embodiment, the substrate may be a premium-grade paper with a controlled thickness of approximately 0.2 mm, offering stability during printing. Further, for wearable applications, a medical-grade thermoplastic polyurethane (TPU) film with a thickness of only 0.08 mm is used. The TPU film is extraordinarily soft, exceptionally lightweight, and capable of stretching by up to 10% without the slightest degradation in circuit integrity. Such elasticity ensures that the finished phototherapy device can conform perfectly to complex body contours without mechanical stress or electrical failure.

The conductive network is then created in step 204 by applying a high-purity, nano-enhanced conductive silver paste to the prepared stencil. Using a precision scraper, the paste is deposited through the fine mesh openings, resulting in a sharply defined printed circuit pattern directly on the substrate surface. Step 206 further produces electrical pathways of exceptional uniformity and adhesion, ready to host the micro-LED lamp beads 102. The method eliminates the need for bulky copper layers and rigid PCB laminates, replacing them with a seamless, ink-based conductor that is both ultra-thin and mechanically forgiving.

With the printed circuitry in place, each micro-LED lamp bead 102 is meticulously positioned on its designated welding pad 120 using high-strength, UV-curable adhesive. This adhesive not only locks the micro-LEDs in perfect alignment but also withstands repeated flexing without detachment. Electrical connectivity between the LED terminals and the printed conductive traces is achieved by depositing a precision bead of conductive silver paste on each pin, ensuring minimal contact resistance and robust long-term durability.

The partially assembled circuit then undergoes a thermal curing process, a finely tuned cycle at approximately 120° C. for 30 minutes. This dual-action step simultaneously activates the UV adhesive's full bonding potential and sinters the conductive silver particles, transforming the printed traces into high-conductivity, mechanically stable interconnects at step 208. The result is an LED-FPCB that is not only operational but also remarkably resilient to mechanical deformation.

When fabricated on TPU or paper substrates, the resulting LED flexible printed circuit board (LED-FPCB) demonstrates effortless illumination with consistent brightness across all micro-LED lamp beads 102. The TPU-based version, in particular, boasts a total thickness of only about 0.08 mm, thinner than a sheet of standard office paper, while retaining the softness of fabric. This means the device can be rolled, folded, or wrapped around body parts with virtually no impact on function.

The TPU film-based LED-FPCB can be produced at a material cost of a staggering 1/40th the cost of conventional FPCBs. This extraordinary reduction in cost opens the door to mass deployment of high-performance wearable phototherapy devices that would otherwise be prohibitively expensive.

The screen-printed circuit fabrication method is optimal for producing ultra-thin, ultra-flexible, and ultra-light LED-FPCBs. The synergy of softness, stretchability, and low weight makes this technology uniquely suited to wearable phototherapy products. The combination of comfort, adaptability, and unprecedented cost efficiency represents a breakthrough in the design and manufacture of therapeutic light-delivery systems, positioning this method as a transformative step in the evolution of personal healthcare technology.

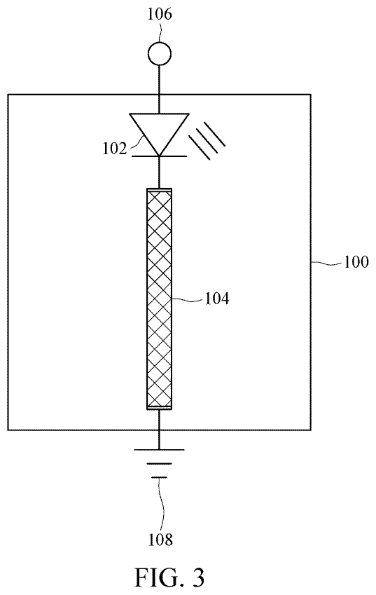

Referring to FIG. 3, a phototherapy device is provided with a circuit board 100, a plurality of micro-LED lamp beads 102 or microlasers, and a metal grid current-limiting structure 104. The micro-LED lamp beads 102 are electrically connected to the circuit board 100, while the metal grid current-limiting structure 104 is configured to limit the current flowing through the micro-LED lamp beads 102. The metal grid current-limiting structure 104 is disposed on the circuit board 100 and is connected in series with micro-LED lamp beads 102 to regulate electrical flow during operation.

In the present embodiment, the circuit board 100 serves as the base structure of the micro-LED lamp beads 102, providing mechanical support and electrical interconnection for components such as the micro-LED lamp beads 102 and the metal grid current-limiting structure 104. The circuit board 100 is preferably fabricated from materials exhibiting excellent insulation performance and high heat resistance, thereby ensuring operational stability and preventing short-circuiting or thermal damage during prolonged use.

The micro-LED lamp beads 102 function as the primary light-emitting elements of the phototherapy device. In an embodiment, the micro-LED lamp beads 102 may be light-emitting diodes that are capable of producing light across various wavelengths to achieve different colour outputs. Such versatility allows the phototherapy device to be applied in a range of uses, including but not limited to beauty treatments and therapeutic light therapy applications.

The metal grid current-limiting structure 104 is a specially engineered component designed to replace conventional resistors for current regulation. The metal grid current-limiting structure 104 can be fabricated from highly conductive materials such as copper or aluminium, which are precision-processed into a predetermined grid pattern. This grid configuration not only ensures precise current control but also facilitates efficient heat dissipation, thereby enhancing the reliability and service life of the lamp board.

In an embodiment, the metal grid current-limiting structure 104 regulates the current flowing through the micro-LED lamp beads 102 by utilizing the inherent electrical resistance of the metallic material from which it is formed. The resistance value of the metal grid current-limiting structure 104 is determined by multiple parameters, including but not limited to the type of metal material, the thickness of the conductive elements, the width of the grid segments, and the overall length of the conductive path. For instance, finer metal wires or smaller mesh apertures yield higher electrical resistance, thereby more effectively limiting current. Moreover, due to its relatively large surface area, the metal grid current-limiting structure 104 facilitates enhanced heat dissipation, maintaining the operating temperature of the micro-LED lamp beads 102 within a safe range and reducing the likelihood of thermal damage.

By employing the metal grid current-limiting structure 104 in place of discrete resistors, the overall circuit design can be simplified, resulting in a more concise and aesthetically streamlined wiring arrangement. As the metal grid current-limiting structure 104 is directly integrated onto the surface of the circuit board 100, spatial efficiency is improved, thereby enabling a more compact phototherapy device construction. The compactness further facilitates ease of installation, handling, and maintenance.

Accordingly, the lamp board provided in the embodiments of the present application achieves multiple technical effects by replacing conventional resistors with the metal grid current-limiting structure 104. Specifically, it ensures precise current regulation for the micro-LED lamp beads 102, improves thermal management, reduces component count, and optimizes circuit layout, thereby enhancing the reliability, durability, and overall appearance of the lamp board.

In an embodiment, the flexible substrate is formed from a transparent or translucent polymeric material, such as medical-grade silicone or polyurethane, that allows the therapeutic light to pass through while maintaining biocompatibility. The substrate incorporates embedded conductive traces configured as a thin-film conductive layer or a printed metal-mesh network, which not only supplies power to the micro-LEDs but also allows optical transparency for underlying light transmission. The flexibility of the substrate enables integration into multiple wearable formats, such as facial masks, wraps, patches, or garment inserts, without compromising the user's mobility or comfort.

In an embodiment, the flexible substrate is configured to conform to the contours of a user's treatment area, such as the face, neck, scalp, or joints. The substrate supports an array of micro-LED lamp beads 102 or micro-lasers that are positioned to deliver uniform and targeted phototherapeutic energy. These light sources are arranged in addressable treatment zones, enabling selective control over the emission wavelength and intensity for different dermatological or therapeutic objectives, such as anti-aging, acne management, pigmentation correction, wound healing, and pain relief. Integrated current-limiting circuitry, such as a metal-grid patterned thin-film conductor, ensures stable operation while minimizing localized heating.

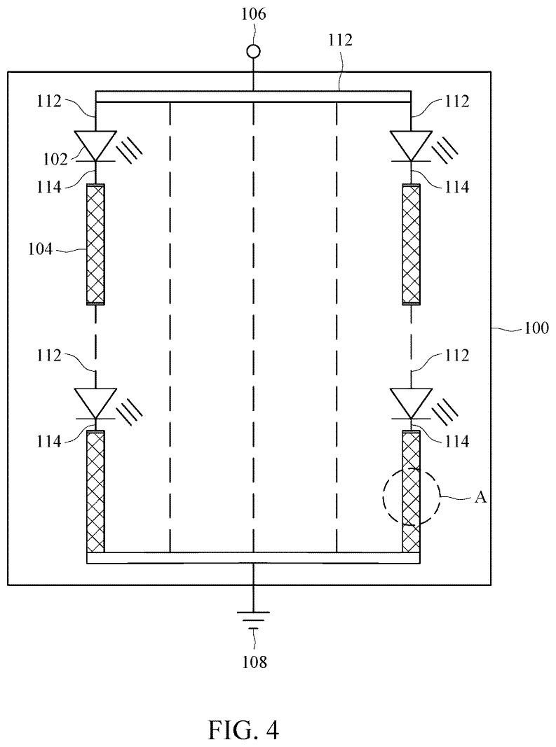

Referring to FIGS. 3 and 4, in an embodiment, the circuit board 100 is provided with a power terminal 106 and a ground terminal 108. The power terminal 106 is configured to be connected to an external power source, while the ground terminal 108 is connected to ground potential. The micro-LED lamp beads 102 and the metal grid current-limiting structure 104 are connected in series between the power terminal 106 and the ground terminal 108, enabling controlled current flow through the micro-LED lamp beads 102.

In the embodiment illustrated in FIG. 4, multiple micro-LED lamp beads 102 are provided and arranged in a plurality of columns. The micro-LED lamp beads 102 within each column are connected in parallel, while the micro-LED lamp beads 102 in each column are sequentially connected in series via the metal grid current-limiting structure 104. This configuration ensures uniform current distribution among the micro-LED lamp beads 102 while maintaining effective current limitation and heat dissipation across the entire lamp board.

In the present embodiment, the micro-LED lamp beads 102 are arranged in multiple columns to form a plurality of phototherapy light boards 110. The micro-LED lamp beads 102 within each column of lamp bead circuits 110 are electrically connected in parallel. Such a configuration ensures that a malfunction in one column of micro-LED lamp beads 102 does not affect the operation of micro-LED lamp beads 102 in other columns, thereby improving fault tolerance.

Within each column of phototherapy light board 110, a corresponding metal grid current-limiting structure 104 is connected in series with the micro-LED lamp beads 102. This arrangement enables the current flowing through each column of micro-LED lamp beads 102 to be independently regulated by its respective metal grid current-limiting structure 104, thereby protecting each column from overcurrent conditions. Furthermore, as the metal grid current-limiting structures 104 are dedicated to individual columns, there is no electrical interference between columns. This independence not only enhances current control but also optimizes the wiring layout of the lamp board, simplifying circuit design and facilitating maintenance.

As will be appreciated, because the micro-LED lamp beads 102 in each column are connected in parallel, the micro-LED lamp beads 102 within a given column share the same supply voltage. When current is supplied to the lamp board, it is evenly distributed among the multiple columns. Consequently, a fault in either the micro-LED lamp beads 102 or the metal grid current-limiting structure 104 of one column will not impair the functionality of the other columns, thereby enhancing the operational reliability of the lamp board as a whole. The metal grid current-limiting structure 104 within each column is configured to precisely regulate the current delivered to the micro-LED lamp beads 102 by selecting appropriate grid dimensions, conductive path geometry, and material composition. This precise current control ensures that all micro-LED lamp beads 102 operate within safe current limits.

In addition to current regulation, the metal grid current-limiting structures 104 serve as auxiliary heat-dissipating elements. Owing to their increased surface area and high thermal conductivity, the metal grids facilitate the transfer of heat away from the micro-LED lamp beads 102, thereby reducing their operating temperature. This thermal management function extends the service life and operational stability of the micro-LED lamp beads 102. Accordingly, the lamp board design of the present embodiment, which incorporates multiple columns of parallel-connected micro-LED lamp beads 102 each with its own metal grid current-limiting structure 104, provides a broader phototherapy coverage area, thereby enhancing therapeutic effectiveness. At the same time, it achieves precise current control, superior heat dissipation, a simplified circuit arrangement, and improved product reliability and aesthetic appeal.

Furthermore, in an embodiment, the circuit board 100 is further provided with a first connecting wire 112 and a second connecting wire 114. The first connecting wire 112 electrically connects one end (for example, the positive electrode) of each column of micro-LED lamp beads 102, thereby linking all columns together to form a common input terminal, which is electrically connected to the power terminal 106. The second connecting wire 114 electrically connects the other end (for example, the negative electrode) of each column of micro-LED lamp beads 102, thereby linking all columns together to form a common output terminal, which is electrically connected to the ground terminal 108. In case of phototherapy devices with a metal-grid current limiting structure, the first connecting wire 112 and the second connecting wire 114 serve as the connection line to the micro-LEDs and the metal-grid current limiting structure is provided between the first connecting wire 112 and the second connecting wire 114 to act as a resistive element. Further, the first connecting wire 112 and the second connecting wire 114 serve as the connection line and are a part of a thin film conductive structure printed on a substrate.

In this arrangement, the inclusion of the first connecting wire 112 and the second connecting wire 114 significantly simplifies the wiring layout on the circuit board 100. Rather than requiring individual routing paths for each column of the micro-LED lamp beads 102, these two connecting wires provide a unified electrical connection for all columns at both ends. This configuration results in a more organized and streamlined wiring arrangement, thereby improving the assembly efficiency, operational reliability, and ease of maintenance of the lamp board. Additionally, the simplified layout enhances the visual appearance of the circuit board 100.

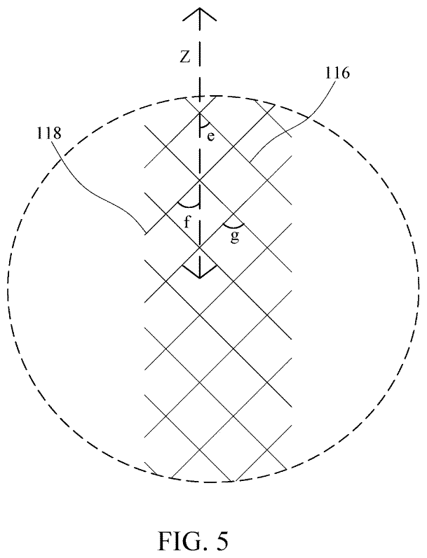

Referring to FIG. 5, in an embodiment, the metal grid current-limiting structure 104 comprises a plurality of first conductive lines and a plurality of second conductive lines 118. The first conductive lines 116 are arranged at equal intervals along the length direction Z of the metal grid current-limiting structure 104, while the second conductive lines 118 are also arranged at equal intervals along the length direction Z of the metal grid current-limiting structure 104. The first conductive lines 116 and the second conductive lines 118 are arranged to intersect with the length direction Z, thereby forming a conductive grid pattern.

Each first conductive line 116 is electrically connected to at least two second conductive lines 118, and each second conductive line 118 is electrically connected to at least two first conductive lines 116. This interconnected grid configuration provides a continuous conductive path with distributed electrical resistance, thereby enabling precise current regulation for the micro-LED lamp beads 102 while also offering enhanced thermal dissipation performance. The structural uniformity of the conductive grid further contributes to the stability, durability, and manufacturing consistency of the metal grid current-limiting structure 104.

In the present embodiment, the first conductive lines 116 are arranged at equal intervals along the length direction Z of the metal grid current-limiting structure 104. These first conductive lines 116 form one of the primary structural frameworks of the metal grid current-limiting structure 104. Correspondingly, the second conductive lines 118 are also arranged at equal intervals along the length direction Z of the metal grid current-limiting structure 104 and are oriented to intersect the first conductive lines 116. The second conductive lines 118 constitute another primary structural framework of the metal grid current-limiting structure 104.

Each first conductive line 116 is electrically connected to at least two second conductive lines 118, and each second conductive line 118 is likewise electrically connected to at least two first conductive lines 116. This staggered interconnection results in a mechanically robust and electrically stable metal mesh or grid configuration. The metal grid current-limiting structure 104 regulates the flow of current by virtue of the inherent resistance of the metallic material. When electrical current passes through the metal mesh, this resistance prevents excessive current flow, thereby protecting the lamp beads (2) from overcurrent damage.

As the metal mesh or grid is composed of multiple intersecting first conductive lines 116 and second conductive lines 118, the current is distributed throughout the entire mesh or grid structure. This distribution facilitates uniform current delivery to the micro-LED lamp beads 102, ensuring that each micro-LED lamp bead 102 operates at an appropriate and safe current level. In addition to its current-regulating function, the metal grid current-limiting structure 104 exhibits high thermal conductivity, enabling it to effectively dissipate heat generated by the micro-LED lamp beads 102. Enhanced heat dissipation efficiency reduces the operating temperature of the micro-LED lamp beads 102, thereby prolonging their service life and improving operational stability.

Furthermore, the integrated metal grid current-limiting structure 104 contributes to a simplified circuit board layout. By replacing conventional discrete resistors with the metal grid design, the overall component count on the circuit board 100 is reduced, resulting in a more concise and organized arrangement. Consequently, the lamp board design of the present embodiment achieves precise current control, improved heat dissipation, reduced component complexity, and enhanced overall reliability through the structural advantages of the metal grid current-limiting structure 104.

This configuration also contributes to simplifying the wiring layout on the circuit board 100, thereby rendering the overall design of the lamp board more concise, organized, and aesthetically appealing.

According to one embodiment of the present application, the number of second conductive lines 118 connected to each first conductive line 116 ranges from two to six, and the number of first conductive lines 116 connected to each second conductive line 118 likewise ranges from two to six. In the present embodiment, the number of first conductive lines 116 corresponds to the number of second conductive lines 118 to form a stable grid structure. Each first conductive line 116 is electrically connected to two to six second conductive lines 118, and each second conductive line 118 is similarly connected to two to six first conductive lines 116. This configuration ensures both the structural stability and the mechanical strength of the metal grid current-limiting structure 104.

By selectively adjusting the number of conductive line connections and the interconnection pattern between the first conductive lines 116 and the second conductive lines 118, the present embodiment can finely control the current distribution across the metal grid current-limiting structure 104. As a result, each micro-LED lamp bead 102 is supplied with an appropriate operating current, thereby achieving a more uniform illumination effect and improving the overall performance of the lamp board.

Referring to FIG. 5, in an embodiment, the angle ‘e’ between the first conductive lines 116 and the lengthwise direction Z of the metal grid current-limiting structure 104 is in the range of 30° to 60°. The angle ‘f’ between the second conductive line 118 and the lengthwise direction Z of the metal grid current-limiting structure 104 is likewise in the range of 30° to 60°. The angle ‘g’ formed between the first conductive lines 116 and the second conductive line 118 is in the range of 60° to 90°. These angular configurations contribute to the formation of a mechanically robust and electrically efficient grid pattern that enhances both current regulation and heat dissipation characteristics.

In the present embodiment, the angle between the first conductive lines 116 and the lengthwise direction Z of the metal grid current-limiting structure 104 is in the range of 30° to 60°. This inclination provides an optimized conductive path that promotes balanced current distribution within the mesh. Similarly, the angle between the second conductive line 118 and the lengthwise direction ‘Z’ of the metal grid current-limiting structure 104 is also in the range of 30° to 60°. The inclined arrangement of the second conductive lines 118 contributes to the formation of a mechanically stable and electrically uniform grid pattern. Furthermore, the angle between the first conductive lines 116 and the second conductive lines 118 is in the range of 60° to 90°, thereby ensuring the mechanical integrity and structural strength of the metal grid current-limiting structure 104. By controlling these angular relationships, the present embodiment achieves optimized current distribution within the metal grid current-limiting structure 104, thereby ensuring uniform current delivery to the micro-LED lamp beads 102 and producing a consistent lighting effect.

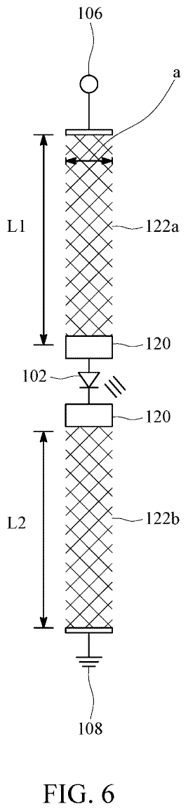

Referring to FIG. 6, in an embodiment, the metal grid current-limiting structure 104 is provided with at least one welding pad 120. The welding pad 120 is configured to be soldered to the electrodes of the micro-LED lamp beads 102, and is electrically connected to at least two adjacent first conductive lines 116 and at least two adjacent second conductive lines 118. This multi-line connection ensures that the current supplied to each micro-LED lamp bead 102 is derived from multiple conductive paths, thereby further enhancing the uniformity of current distribution and the consistency of light emission across the lamp board.

Additionally, as the welding pads 120 are physically and thermally coupled to the first metal grid current-limiting structure 122a and second metal grid current-limiting structure 122b, they also facilitate heat transfer from the micro-LED lamp beads 102 into the metal grid. This enables the first metal grid current-limiting structure 122a and second metal grid current-limiting structure 122b to function not only as a current regulator but also as a heat sink, thereby improving the thermal dissipation efficiency of the entire lamp board. The provision of a welding pad 120 integrated with the first metal grid current-limiting structure 122a and second metal grid current-limiting structure 122b also simplifies the wiring layout on the circuit board 100, reducing the need for additional conductive traces and resulting in a more concise and organized circuit arrangement.

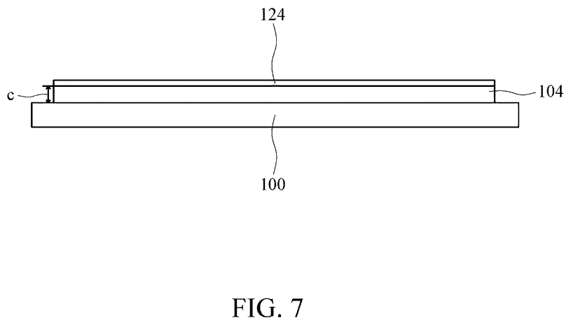

Referring to FIGS. 6 and 7, in an embodiment, the phototherapy light board 110 comprises two identical metal grid current-limiting structures 104: a first metal grid current-limiting structure 122a and a second metal grid current-limiting structure 122b. One end of the first metal grid current-limiting structure 122a is electrically connected to the power terminal 106, and the opposite end, second metal grid current-limiting structure 122b, is connected to the positive electrode of the micro-LED lamp beads 102 through a welding pad 120. Likewise, one end of the second metal grid current-limiting structure 122b is connected to the negative electrode of the micro-LED lamp beads 102 via a welding pad 120, while the opposite end is connected to the ground terminal 108.

The total resistance R of the two metal grid current-limiting structures in the phototherapy light board 110 is calculated according to the following relationship:

$$R = K \times ρ \times L / ( a \times c ) ; \\ L = L 1 + L 2 ;$$

where: R represents the total resistance of the two metal grid current-limiting structures; K denotes the mesh duty cycle, defined as the ratio of the total open mesh area to the total area of the two metal grid current-limiting structures; ρ is the metal resistivity; L is the total length of the two metal grid current-limiting structures; L₁ is the length of the first metal grid current-limiting structures; L2 is the length of the second metal grid current-limiting structures; a is the width of the metal grid current-limiting structures; and c is the thickness of the metal grid current-limiting structures.

Referring to FIG. 7, in an embodiment, the circuit board 100 is provided with an insulating layer 124. The insulating layer 124 covers the metal grid current-limiting structures 104 while defining an opening to expose the welding pad 120. This configuration ensures electrical insulation while allowing a reliable electrical connection to the lamp bead electrodes.

In this embodiment of the present application, the inclusion of the insulating layer 124 ensures reliable electrical isolation between the metal grid current-limiting structure 3 and other conductive elements present on the circuit board 100. This arrangement effectively prevents the occurrence of short circuits, thereby enhancing operational safety and reliability of the light board.

The insulating layer 124 is provided with an opening configured to expose the welding pad 120. This structural feature facilitates direct soldering of the electrodes of the micro-LED lamp beads 102 onto the welding pad 120, thereby simplifying the assembly process. Additionally, the exposed welding pad 120 ensures a secure and stable electrical connection, which contributes to the overall durability and consistent performance of the device.

The insulating layer 124 may be formed from materials possessing excellent dielectric properties, such as polyimide film, polyester film, or other high-performance polymeric materials, thereby ensuring long-term insulation stability under varying operating conditions, while maintaining flexibility and, in certain configurations, optical transparency. In some embodiments, the insulating layer may be formed from thermoplastic polyurethane (TPU) or similar elastomeric insulating films to enable stretchability for wearable applications. The choice of insulating material is selected based on the required combination of dielectric strength, thermal resistance, transparency, and mechanical flexibility to suit the intended phototherapy device configuration.

According to one embodiment of the present application, the micro-LED lamp beads 102 or microlaser are light-emitting diodes. By employing micro-LED lamp beads 102 in combination with the metal grid current-limiting structure 104 and the insulating layer 124, the present application provides a light board that delivers highly effective beauty and skincare treatment benefits, while simultaneously achieving extended service life, superior energy efficiency, and excellent operational stability.

According to one embodiment of the present application, the circuit board 100 may be configured as either a flexible or a rigid insulating layer 124.

This versatility enables the light board to be adapted for various phototherapy devices. For example, when the circuit board 100 is implemented as a flexible base 126, the resulting light board can be incorporated into a phototherapy facial patch as illustrated in FIG. 1B. Conversely, when the circuit board 100 is implemented as a base 126, the resulting light board can be integrated into a phototherapy device as shown in FIG. 1C.

In one embodiment, the metal grid current-limiting structure 3 is formed as a metal layer plated directly onto the circuit board 100. The metal grid current-limiting structure 3 may be deposited via processes such as vacuum sputtering or electroplating, ensuring that the structure is tightly bonded to the circuit board 100. This integrated fabrication method minimizes the need for additional connectors, thereby simplifying the circuit layout. Moreover, by forming the metal grid current-limiting structure 3 through plating, the number of connection points is reduced, which lowers the risk of electrical failure and enhances both the structural stability and long-term reliability of the light board.

As will be appreciated, traditional resistors typically occupy a substantial volume, particularly in high-power applications, which constrains the miniaturization and compactness of phototherapy devices.

In contrast, the layered metal grid current-limiting structure 104 employed in the present application significantly reduces the spatial footprint required for current regulation. This compact design frees up valuable space for other components within the phototherapy device, thereby enhancing overall space utilization. Consequently, the phototherapy device can be designed to be more compact, lightweight, and portable without compromising performance.

In an embodiment, a phototherapy device is provided that incorporates the light board as described in the foregoing embodiments. The specific type of phototherapy device is not particularly limited.

For example, the phototherapy device may be a phototherapy facial patch as illustrated in FIG. 1B. The phototherapy facial patch includes a base 126 and a light panel disposed therein. The rear side of the base 126 may include a transparent sheet 128. The light panel may be implemented as a flexible light panel located within the base 126. Light emitted from the light panel passes through the transparent sheet 128 to provide targeted phototherapy skin care for the face.

Alternatively, the phototherapy device may correspond to the phototherapy device shown in FIG. 1C, which includes a housing 130 and a light-transmitting plate 132 disposed within the housing 130. The front side of the housing 130 includes a light-transmitting plate 132 through which light emitted by the phototherapy light board 110 is transmitted. This arrangement facilitates phototherapy skin care for various parts of the body.

In an embodiment, the phototherapy device further comprises a charging connector assembly that enables a reliable electrical connection between an external power source and the internal light therapy circuitry. The charging connector incorporates a specially designed cable clip and connecting body arrangement that securely couples the charging connector to the lamp board within the device housing. This arrangement not only facilitates efficient power transmission for charging and operation but also provides enhanced mechanical support to prevent loosening, cable disconnection, or solder joint damage during repeated use. By combining cable-retention features with structural engagement between the connector assembly and the light board, the invention ensures a stable, durable, and safe charging interface for phototherapy applications.

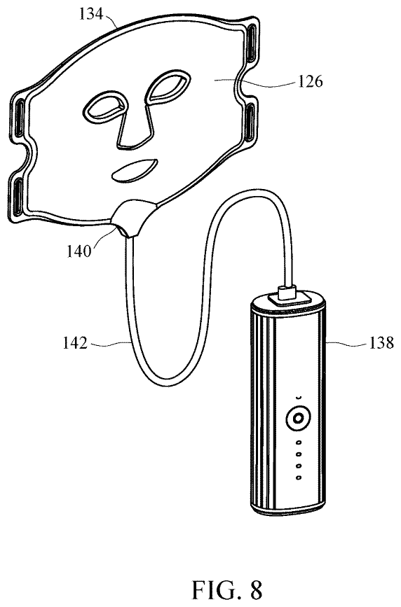

Referring to FIGS. 8 to 10, in an embodiment, a phototherapy device is disclosed comprising a mask 134, a cable clip 136, and a cable 142. The mask 134 includes a transparent sheet 128 and a phototherapy light board 110. The transparent sheet 128 is provided with a wire hole 140, within which the cable clip 136 is positioned. The phototherapy light board 110 is mounted inside the transparent sheet 128, and the cable 142 is configured to electrically connect the phototherapy light board 110 to a control box 138. One end of the cable 142 passes through the wire hole 140, is inserted into the cable clip 136, and is electrically connected, preferably by soldering, to the phototherapy light board 110. The cable clip 136 serves to fix and stabilize the end of the cable 142 within the wire hole 140.

In an embodiment, the phototherapy mask 134 is formed of flexible silicone, providing both comfort and adaptability to a user's facial contours. The transparent sheet 128 constitutes the main structural component, supporting and protecting the internal elements of the device. The wire hole 140 is dimensioned to allow passage of the cable 142, thereby serving as an installation channel.

The phototherapy light board 110 constitutes the primary functional element of the device and is configured to deliver cosmetic and skin care benefits. The phototherapy light board 110 carries multiple micro-LED lamp beads 102 capable of emitting light at specific wavelengths, such as red light, blue light, or other suitable therapeutic spectra. These wavelengths may provide targeted treatment effects for various skin conditions, such as acne reduction, anti-aging, and skin tone improvement. The cable clip 136 may be either movably or fixedly retained within the wire hole 140 of the transparent sheet 128, and its function is to prevent the cable 142 from being pulled out of the transparent sheet 128.

In the preferred configuration, the soldering point 144 between the cable 142 and the phototherapy light board 110 is reinforced to minimize the risk of detachment under mechanical stress. The wire hole 140 is shaped such that its cross-sectional area gradually decreases from the end closest to the mask 134 interior toward the exterior end. The cable clip 136 has a cross-sectional area larger than the narrowest portion of the wire hole 140, thereby ensuring that the cable clip 136 remains securely retained within the wire hole 140 while permitting limited movement to relieve tension.

The cable 142 may be removably connected to the control box 138. In operation, the cable 142 supplies electrical power from the control box 138 to the phototherapy light board 110, and also transmits control signals to adjust treatment parameters. The user may operate the control box 138 to select among various treatment modes, which may include different light colours, intensities, and durations. When the control box 138 is activated, current is delivered to the micro-LED lamp beads 102 via the cable 142, causing them to emit light of the selected wavelength.

The use of the cable clip 136 significantly enhances the stability of the electrical connection between the cable 142 and the phototherapy light board 110, maintaining reliable electrical contact even when the device is subjected to frequent movement or minor pulling during daily use. This structural arrangement ensures both operational safety and extended service life. Furthermore, the provision of an external control box 138 allows the user to customize treatment protocols according to individual skin conditions and personal preferences, thereby improving the versatility, comfort, and overall user experience of the phototherapy mask 134.

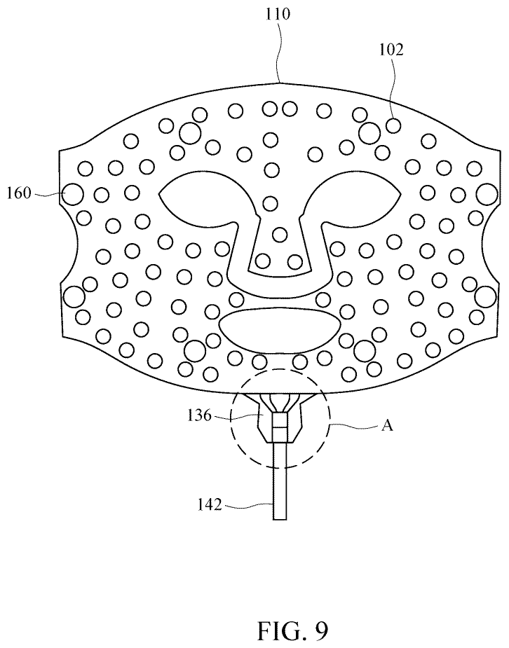

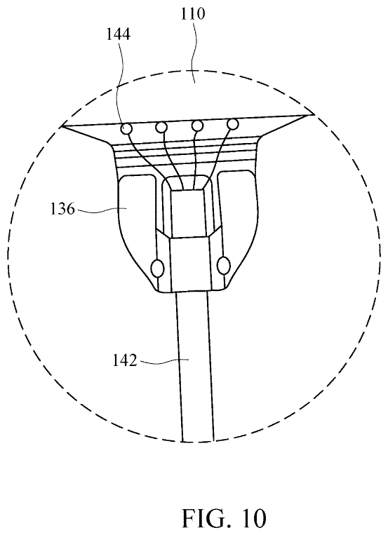

Referring to FIGS. 9 to 10, in an embodiment, the cable clip 136 comprises a cable clip body 146. The cable clip body 146 is formed with a cable clamping channel 150 extending therethrough. An inner wall of the cable clamping channel 150 is provided with a convex ring 152. One end of the cable 142 is inserted through the cable clamping channel 150 such that the convex ring 152 engages with the outer surface of the cable 142 to securely clamp it in place, thereby preventing undesired loosening or displacement of the cable 142 along the axial direction of the cable clamping channel 150.

In the illustrated embodiment, the cable clip body 146 serves as the primary structural element of the cable clip 136 and is specifically designed to accommodate and fix the cable 142. The cable clamping channel 150 is dimensioned to allow the cable 142 to pass through and subsequently connect to the phototherapy light board 110 within the transparent sheet 128 of the mask 134.

The convex ring 152 is formed as an annular protrusion on the inner wall of the cable clamping channel 150. This structural feature provides a mechanical interference fit with the cable 142 as it passes through the channel, thereby creating a physical barrier that resists axial movement of the cable 142. This arrangement effectively prevents the cable 142 from sliding out of the cable clamping channel 150 or undergoing unnecessary displacement when subjected to external forces.

By maintaining the cable 142 in a fixed position relative to the cable clip 136, the convex ring 152 reduces mechanical stress on the soldered connection between the cable 142 and the phototherapy light board 110. This, in turn, lowers the risk of detachment or damage caused by repeated pulling or movement during use. The arrangement ensures stable current transmission from the control box 138 to the phototherapy light board 110, thereby enhancing both the reliability and durability of the phototherapy mask 134 during long-term operation.

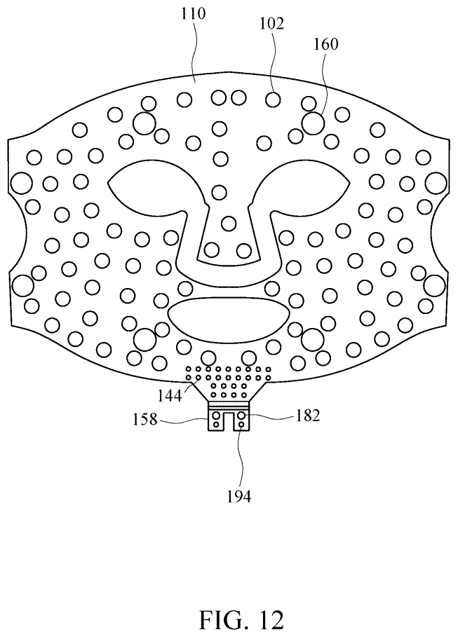

Referring to FIGS. 10 to 12, in an embodiment, the cable clip 136 further comprises a connecting body 156. The connecting body 156 is integrally formed with, or otherwise secured to, opposite sides of the cable clip body 146. A portion of the phototherapy light board 110 adjacent to the cable clip 136 is provided with a fixing portion 158 that is correspondingly connected to the connecting body 156.

In an embodiment, the connecting body 156 is positioned at the two opposing sides of the cable clip body 146 and is configured to mechanically couple with the fixing portions 158 formed on the phototherapy light board 110. The fixing portions 158 may be provided with fastening holes 160, which are adapted to receive screws, bolts, or other mechanical fasteners. During regular use, any tensile force applied to the wiring is not directly transmitted to the solder joints between the wires and the lamp board due to the presence of the fastening holes 160. Instead, the force is borne by the connection between the lamp board and the line card. Furthermore, the lamp board is supported and stabilized within the silicone mask/phototherapy device, thereby providing additional protection to the solder joints. This structural arrangement ensures that pulling on the wires during everyday use does not result in malfunction or damage to the solder connections. This arrangement allows the phototherapy light board 110 to be securely fixed within the transparent sheet 128 of the mask 134. By engaging the connecting body 156 of the cable clip 136 with the fixing portions 158 of the phototherapy light board 110, the cable clip 136 becomes an integral part of the structural support for the phototherapy light board 110, thereby increasing its rigidity and structural integrity.

Functionally, the cable 142 is held in position within the cable clip 136 by means of the convex ring 152 and, in some embodiments, by an additional clamping strip 154. At the same time, the cable clip 136 is mechanically secured to the phototherapy light board 110 through the connecting body 156 and the fixing portions 158. This dual securing mechanism ensures that the cable 142 maintains a stable position within the overall device assembly.

Once the cable clip 136 and the phototherapy light board 110 are firmly joined, they form a unified structure that significantly reduces the likelihood of cable 142 displacement or solder point 144 loosening due to external forces. This design thereby enhances the mechanical stability and electrical reliability of the connection between the cable 142 and the phototherapy light board 110, prolonging the operational life of the device.

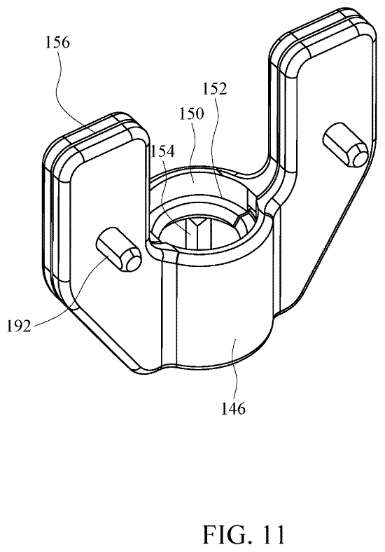

Referring to FIG. 11, in an embodiment, the number of convex rings 152 provided on the inner wall of the cable clamping channel 150 is at least two, with the convex rings 152 being arranged at intervals along the axial direction of the cable clamping channel 150. When two or more convex rings 152 are disposed within the cable clamping channel 150, multiple clamping points are formed along the length of the cable 142, thereby improving its positional stability. The spaced-apart arrangement of the convex rings 152 allows the tensile load on the cable 142 to be distributed across different positions, reducing the likelihood of localized stress damage.

In one specific configuration, a convex ring 152 is positioned at the entrance and another at the exit of the cable clamping channel 150. This arrangement ensures that the cable 142 is clamped both upon entry and upon exit, thereby forming a closed-loop clamping mechanism. In certain embodiments, the convex rings 152 may be manufactured from an elastic material or be provided with a degree of resilience, allowing them to accommodate the cable 142 of varying diameters, thereby enhancing the adaptability and versatility of the product. By employing multiple convex rings 152, the mechanical clamping force is improved, the electrical connection reliability is enhanced, and the operational service life of the phototherapy mask 134 is extended.

In an embodiment, the inner wall of the cable clamping channel 150 is further provided with at least one clamping strip 154 extending along the axial direction of the channel. The clamping strip 154 serves to restrict the rotation of the cable 142 about the axial direction of the cable clamping channel 150. In the illustrated embodiment, the convex rings 152 and the clamping strip 154 cooperate to form a three-dimensional securing structure around the cable 142, wherein the convex rings 152 primarily limit axial movement (insertion and withdrawal) and the clamping strip 154 prevents circumferential rotation.

The presence of the clamping strip 154 ensures that the cable 142 remains upright and correctly oriented within the cable clamping channel 150, thereby preventing twisting that could otherwise lead to conductor fatigue or solder point 144 loosening. In certain embodiments, the clamping strip 154 may also possess a degree of elasticity, enabling it to adapt to different cable thicknesses. When the cable 142 is inserted into the cable clamping channel 150, the convex rings 152 form multiple fixed contact points to prevent axial sliding, while the clamping strip 154 is embedded into the surface texture of the cable 142 or presses against it with sufficient force to prevent rotation. This combined structure provides a robust securing mechanism that resists both tensile and torsional forces, thereby reducing the risk of damage due to improper handling or external mechanical stress.

Furthermore, the number of clamping strips 154 is at least two. The clamping strips 154 are arranged at circumferentially spaced intervals along the inner wall of the cable clamping channel 150. By distributing the clamping strips 154 at different angular positions, the contact pressure applied to the cable 142 is spread evenly, thereby avoiding localized stress concentration. Furthermore, the multi-directional clamping provided by the circumferentially spaced clamping strips 154 prevents rotational movement of the cable 142 and ensures that it remains in a straight, aligned position. This configuration not only improves the mechanical and electrical connection stability between the cable 142 and the phototherapy light board 110 but also enhances the overall durability, usability, and user experience of the phototherapy mask 134.

Referring to FIGS. 11, 13, and 14, in an embodiment, the cable clip 136 is formed by splicing together a first card body 162 and a second card body 164. The first card body 162 includes a first wire clamping portion 166, while the second card body 164 includes a second wire clamping portion 168. When assembled, these portions define a cable clamping channel 150 between them.

The inner wall of the first wire clamping portion 166 is provided with a first ring body 170, and the inner wall of the second wire clamping portion 168 is provided with a second ring body 172. When the first and second card bodies are spliced together, the first ring body 170 and second ring body 172 jointly form a convex ring 152, which engages the outer surface of a cable 142 to provide axial fixation.

Because the cable clip 136 is composed of two separable parts, the cable 142 may be placed in one half first, after which the other half is assembled. This eliminates the need to thread the cable through a completely enclosed structure, thereby reducing assembly difficulty, shortening assembly time, and improving production efficiency.

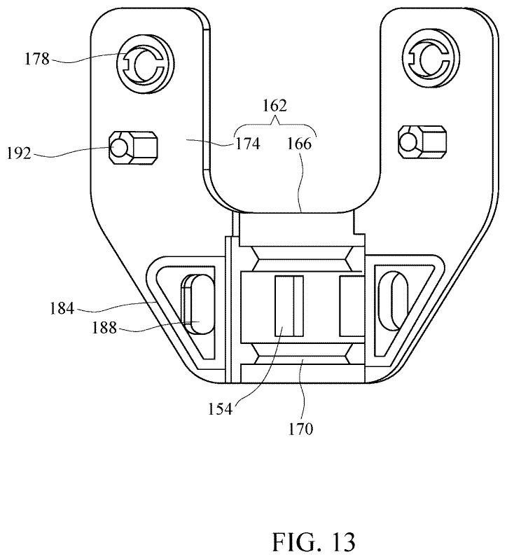

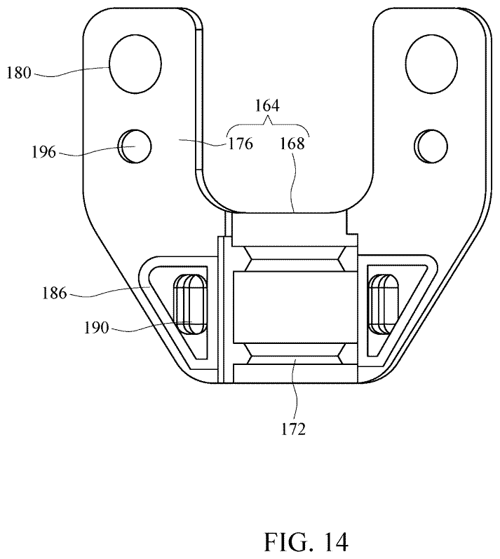

Referring to FIGS. 13 and 14, in an embodiment, the inner wall of the first wire clamping portion 166 and/or the second wire clamping portion 168 is further provided with at least one clamping strip 154. Upon assembly, the clamping strip 154 presses against the cable 142, applying a holding force that effectively restricts circumferential rotation and enhances the positional stability of the cable.

The first card body 162 further includes first connecting portions 174 located on opposite sides of the first wire clamping portion 166. Correspondingly, the second card body 164 includes second connecting portions 176 located on opposite sides of the second wire clamping portion 168. Each first connecting portion 174 is provided, at one end, with a first welding member 178, while each second connecting portion 176 is provided with a first welding groove 180. Each fixing portion 158 of a phototherapy light board 110 is provided with a welding hole 182.

During assembly, the cable 142 is placed within the cable clamping channel 150 and secured by the combined action of the convex ring 152 and the clamping strip 154. The fixing portion 158 of the phototherapy light board 110 is positioned between the first connecting portion 174 and the second connecting portion 176 so that the welding holes 182 align with the corresponding first welding members 178 and first welding grooves 180. An ultrasonic welding device is then used to melt the first welding member 178, causing the molten material to fill the welding hole 182 and the first welding groove 180, thereby creating a high-strength, integrated joint between the cable clip 136 and the phototherapy light board 110.

At the opposite ends of the first connecting portions 174 and second connecting portions 176, second welding members 184 are provided within corresponding second welding grooves 186. Each second welding member 184 includes a positioning hole 188, while each second welding groove 186 includes a positioning column 190 sized to fit into the positioning hole 188. This arrangement ensures accurate alignment of the two card bodies prior to welding. Ultrasonic welding is similarly applied to the second welding members 184 so that they melt and fill the second welding grooves 186, securely bonding the first and second card bodies together and further enhancing the overall structural strength of the cable clip 136.

Moreover, each first connecting portion 174 is provided with a rivet column 192 located centrally relative to the fixing portion 158 of the phototherapy light board 110. Each fixing portion 158 is provided with a first rivet hole 194, and each second connecting portion 176 is provided with a second rivet hole 196. During assembly, the rivet column 192 passes through the first rivet hole 194 and into the second rivet hole 196. A riveting tool is then used to deform and fasten the rivet column 192, forming a mechanical lock that further reinforces the connection between the cable clip 136 and the phototherapy light board 110.

By employing a dual fixing method, ultrasonic welding combined with mechanical riveting, the invention achieves a robust and reliable connection between the cable 142 and the phototherapy light board 110. This structure not only ensures high connection strength and positional stability but also improves product reliability by preventing loosening of the cable, maintaining electrical contact integrity, and reducing the likelihood of solder joint failure during the service life of the product.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A phototherapy device comprising:
a substrate;
a plurality of light-emitting elements disposed on the substrate; and
a thin film conductive structure electrically connecting the plurality of light-emitting elements,
wherein the plurality of light-emitting elements is configured to emit light over a treatment area for phototherapeutic treatment.

2. The phototherapy device of claim 1, wherein the light-emitting elements comprise micro-LEDs or micro-lasers.

3. The phototherapy device of claim 1, wherein the substrate is formed of a stretchable material configured to allow stretching of the device.

4. The phototherapy device of claim 1, wherein the thin film conductive structure comprises a zig-zag wire pattern configured to maintain circuit integrity during stretching.

5. The phototherapy device of claim 1, wherein the substrate is made of a transparent material.

6. The phototherapy device of claim 1, further comprising an outer layer and an inner layer, each formed of a flexible transparent material to provide bendability and transparency.

7. The phototherapy device of claim 6, wherein the substrate and the outer layer are sealed together by vacuum packaging.

8. The phototherapy device of claim 2, wherein the micro-LEDs are arranged in a matrix configuration comprising a plurality of rows and columns.

9. The phototherapy device of claim 1, wherein the device is formed as a facial mask configured to conform to a human face, with openings for eyes, nose, and mouth.

10. The phototherapy device of claim 1, further comprising a pair of external pins configured to connect to an external controller or charging unit via magnetic or wired connection.

11. The phototherapy device of claim 10, further comprising a coupling structure attached to the substrate, the coupling structure being configured to connect the phototherapy device to the external controller or charging unit.

12. The phototherapy device of claim 11, wherein the coupling structure comprises a pair of arms, a central recess, and one or more fastening elements configured to securely mount and allow adjustable positioning of the phototherapy device.

13. The phototherapy device of claim 1, wherein the thin film conductive structure comprises a current supply line and a metal grid current limiting structure.

14. The phototherapy device of claim 13, wherein the metal grid current limiting structure is configured to limit current supplied to the light-emitting elements.

15. A phototherapy device comprising:
(a) a substrate;
(b) a plurality of light-emitting elements disposed on the substrate; and
(c) a thin film conductive structure electrically connecting the plurality of light-emitting elements,
wherein the thin film conductive structure comprises a metal grid current limiting structure electrically connected with at least one of the light-emitting elements, and
wherein the metal grid current limiting structure is configured to limit current flow and enhance thermal dissipation.

16. The phototherapy device of claim 15, wherein the metal grid current limiting structure comprises a plurality of first conductive lines and a plurality of second conductive lines intersecting to form a grid.

17. The phototherapy device of claim 16, wherein each of the first conductive lines is electrically connected to at least two of the second conductive lines, and each of the second conductive lines is electrically connected to at least two of the first conductive lines.

18. The phototherapy device of claim 16, wherein an angle formed between the first conductive lines and the second conductive lines is in range of 60 degrees to 90 degrees.

19. A method of manufacturing a phototherapy device, comprising:
(a) providing a flexible substrate;
(b) forming a circuit pattern for light-emitting elements;
(c) depositing a conductive material onto the flexible substrate in accordance with the circuit pattern, thereby forming a thin film conductive structure on the substrate;
(d) attaching a plurality of light-emitting elements to the thin film conductive structure; and
(e) electrically connecting the light-emitting elements to the thin film conductive structure using a conductive material.

20. The method of claim 19, wherein depositing the conductive material comprises a process selected from the group consisting of screen printing, sputtering, and electroplating.

21. The method of claim 19, wherein the flexible substrate is selected from the group consisting of thermoplastic polyurethane (TPU).

22. The method of claim 19, wherein the light-emitting elements are attached using a UV-curable adhesive.
